(19) United States
(12) Patent Application Publication
(10) Pub. No.: US 2008/0213236 A1
(43) Pub. Date: Sep. 4, 2008

(54) Natural Remedy-Dietary Supplement Combination Product

(76) Inventor: Dana F. Flavin-Koenig, Greenwich, CT (US)

Correspondence Address: GREENBLUM & BERNSTEIN, P.L.C., 1950 ROLAND CLARKE PLACE, RESTON, VA, 20191, US

(21) Appl. No.: 11/815,432

(22) PCT Filed: Feb. 3, 2006
(86) PCT No.: PCT/EP06/00962

§ 371 Date: Aug. 2, 2007

(30) Foreign Application Priority Data
Feb. 3, 2005 (DE) 10 2005 005 086.7

(51) Int. Cl. A61K 33/04; A61K 36/324; A61K 35/66; A61K 35/60; A61P 29/00; A61P 19/02

(52) U.S. Cl. 424/93.51; 424/702; 424/725; 424/523

(57) ABSTRACT

A natural remedy-dietary supplement combination product which comprises omega-3 fatty acids, α-tocopherol, ascorbic acid, selenium, Harpagophytum procumbens (devil's claw) and Boswellia serrata or carterii (frankincense), with the proviso that it does not comprise a plant or plant extract of the Saxifragaceae family, and a method of treating a chronic inflammatory disorder and/or a rheumatic disorder.

The invention relates to a combination product comprising natural remedies and dietary supplements and to the use thereof in the treatment of chronic inflammatory and/or rheumatic disorders, especially of rheumatoid arthritis (chronic polyarthritis).

Rheumatoid arthritis is an inflammatory joint disorder which may lead to serious impairments in daily life. It is the commonest rheumatic disorder and affects about 0.5% of the population, women three times as often as men. It affects in particular the finger joints where, in the initial stage of the disease, it causes pain at night and in the morning and prolonged joint stiffness. Possible later occurrences are involvement of further joints, joint deformations and rarely organ involvement. In a later stage, the so-called pannus, a tumor-like tissue which, after a certain time, destroys cartilage, bone and also other structures of the affected joint develops from the inflamed synovial membrane (lining of the joint).

The reason for the development of rheumatoid arthritis is still not definitively clarified. Genetic factors and autoimmune reactions are probably involved therein. As the disease progresses further, inflammation of the synovial membrane, involving almost all the cells of the immune system and the synovial cells, occurs. This inflammation is controlled mainly by the cytokine tumor necrosis factor α (TNFα), whereas the cytokine interleukin 1 (IL-1) is responsible for the destruction of cartilage tissue and activation of the bone-destroying osteoclasts. The natural antagonist of IL-1, the IL-1 receptor antagonist IL-1Ra, is present in insufficient quantity in rheumatoid arthritis.

Medical therapy of rheumatoid arthritis is currently based primarily on so-called basic therapeutics, which include cytostatics (e.g. methotrexate), immunosuppressants (e.g. cyclosporin A) and gold preparations. However, these basic medicaments have an effect only after weeks or months.

Very recently, inhibitors of TNFα (infliximab and etanercept) and the recombinant IL-1 receptor antagonist anakinra have been administered parenterally as basic therapeutics.

Further substantial supports of the medical therapy of rheumatoid arthritis are the glucocorticoids which rapidly inhibit inflammation and alleviate pain. Also employed are non-steroidal antiinflammatory drugs (NSAID) (e.g. diclofenac and indomethacin), which are antiinflammatory analgesics (antiphlogistics) that inhibit the enzyme cyclooxygenase which is necessary for prostaglandin production. Cyclooxygenase-2 (COX-2)-selective NSAIDs which inhibit only the production of the prostaglandins involved in the inflammatory event but not the protective prostaglandins are a new NSAID class.

Rheumatoid arthritis shows a gradual progression in most cases. Although the progression can often be slowed down, and the inflammation and pain controlled well over a long period by medicaments, nevertheless there is a certain risk of permanent invalidity.

All the medicaments mentioned above for the treatment of rheumatoid arthritis have more or less severe side effects. It would be extremely desirable to find a medicament or a combination of medicaments that acts at least as well as the therapy with the abovementioned synthetic medicaments but which has substantially fewer or even no side effects.

US 2004/0161478 A1 discloses as an agent for the prevention and treatment of arthritis a plant of the family Saxifragaceae or an extract thereof. A pharmaceutical composition with this agent may comprise as further ingredients among many other active ingredients recited selenium, oxidation-preventing vitamin, eicosapentaenoic acid, Boswellia and devil's claw. However, the only demonstration of an effect indicated was a preventive effect in an animal model of arthritis.

Accordingly, the object of the present invention is to find a medicament or a combination of medicaments with the abovementioned desirable properties.

This object is achieved by a natural remedy-dietary supplement combination product which comprises omega-3 fatty acids, α-tocopherol (vitamin E), ascorbic acid (vitamin C), selenium, Harpagophytum procumbens (devil's claw) and Boswellia serrata or carterii (frankincense).

The invention further comprises the use of an inventive natural remedy-dietary supplement combination product in the treatment of inflammatory chronic and/or rheumatic diseases, especially rheumatoid arthritis.

In a further embodiment the invention comprises a method of treatment by administering the inventive natural remedy-dietary supplement combination product to an individual suffering of inflammatory chronic and/or rheumatic diseases, especially rheumatoid arthritis.

It has been found that the inventive combination of dietary supplements and natural remedies exhibits a surprising synergistic effect in rheumatoid arthritis which could not have been predicted on the basis of the effect of the individual components and is of equal quality to that of a conventional medical therapy, but with the great advantage that only few or no side effects are observed.

A. Omega-3 Fatty Acids

There are a number of studies on the effect of omega-3 fatty acids (eicosapentaenoic acid and docosahexaenoic acid) in rheumatoid arthritis. The main source of omega-3 fatty acids is fish oil. It has been found in some studies that about 3 to 5 g of omega fatty acids from fish oil markedly reduce the levels of the markers of an immune dysfunction—IL-1β, IL-6 and TNFα—which are raised in patients with rheumatoid arthritis, and the production of prostaglandin E2 (PGE2) and leukotriene B4 (see, for example, A. Colin, J. Reggers et al. “Lipids, depression and suicide” Encephale 29(1), pp. 49-58 (2003) and literature cited therein; R. I. Sperling et al., “Effects of dietary supplementation with marine fish oil on leukocyte lipid mediator generation and function in rheumatoid arthritis” Arthritis Rheum. 1987, 30. Sep. (9), p. 988-97). Moreover, the production of antiinflammatory (antiphlogistic) substances such as prostaglandin E1 is promoted by omega-3 fatty acids.

It has been possible to show in several studies that this improvement in immune function is associated with less joint pain, less dependence on NSAID and, in some patients, with an improved overall assessment of the disease. Omega-3 fatty acids are prone to oxidation. Studies in humans and mice have shown that vitamin E, which is a potent lipid-soluble antioxidant, appears to contribute to keeping the omega-3 fatty acids unharmed. Vitamin E alone in high doses (1200 mg/day) led in several studies to an alleviation of pain, without influencing the immune markers, although the mechanism of action remained unclear; see, for example, S. Tidow-Kebritchi, S. Mobarhan, Effects of diets containing fish oil and Vitamin E on rheumatoid arthritis. Nutrition Reviews, 2001, vol. 59, pp. 335-338. The authors of this review article remark, however, that the fish oil therapy will probably not become the first-line treatment of rheumatoid arthritis.

B. α-Tocopherol (Vitamin E)

α-Tocopherol (vitamin E) is, as already mentioned, a potent lipid-soluble antioxidant which inhibits the formation of oxygen free radicals from NADPH oxidase in leukocytes and macrophages, which are crucially involved in the cascade of inflammation in arthritis. Human synovial tissue has no natural antioxidant protection in the form of superoxide dismutase, catalase and glutathione peroxidase. The abovementioned pain-relieving effect of vitamin E in rheumatoid arthritis may derive from the capture of reactive oxygen species and the antiinflammatory effect associated therewith in rheumatoid arthritis.

C. Ascorbic Acid (Vitamin C)

The inventive combination product therefore also comprises ascorbic acid (vitamin C) which is well known to be a water-soluble antioxidant which cooperates synergistically with vitamin E in reducing the formation of oxygen free radicals and damage on the level of the cell membrane (inflammation).

D. Selenium (e.g. in the Form of Sodium Selenite, Selenomethionine, Selenoallylcysteine, Selenium Yeast, etc.)

The inventive combination product comprises a selenium compound as further ingredient active against reactive oxygen species (hydroperoxides). Selenium is bound in vivo to the enzyme glutathione peroxidase. This enzyme is able to break down the hydroperoxide products from arachidonic acid, e.g. 5-HPETE, 12-HPETE and 15-HPETE, and thus reduce the concentrations of the proinflammatory secondary products thereof, prostaglandin E2, leukotriene B4 and lipoxin, with a simultaneous promotion of the formation of antiinflammatory eicosanoids from eicosapentaenoic acid, e.g. leukotriene B5, prostaglandin E3 and thromboxane A3.

There are studies which show that selenium concentrations in erythrocytes of patients with rheumatoid arthritis are significantly lower than in the average population. On administration of selenium as adjuvant the patients showed less painful or swollen joint and less morning stiffness, so that less NSAID or cortisol were required (see, for example, K. Heinle et al. “Selenium concentration in erythrocytes of patients with rheumatoid arthritis. Clinical and laboratory chemistry markers during administration of selenium.” Med. Klin. 92, Suppl. 3, pp. 29-31 (Sep. 15, 1997).

E. Harpagophytum procumbens (Devil's Claw)

Harpagophytum procumbens (devil's claw) is a medicinal plant indigenous to southern Africa. The secondary roots are used pharmaceutically and are either prepared as tea or extracted to obtain a dry extract. The roots contain the so-called iridoid glycosides, including harpagoside and harpagide, and inter alia flavonoids, phenolic acids, quinones, phytosterols, sugars, triterpenes and acetoside.

There are numerous studies relating to the antiinflammatory and analgesic effect of subcutaneously injected extract, orally administered extract and tea from the roots of Harpagophytum. The results are contradictory. Whereas some researchers reported an excellent antirheumatic effect in animals and in humans both on oral and on parenteral administration, others were unable to detect any such on oral administration of an extract. Possible reasons cited for the different results were a lack of standardization of the Harpagophytum extracts, but also the possibility that the extracts are inactivated by gastric acid.

Harpagoside has also been investigated as single substance. It was found that it is far less effective than the total root extract.

The mechanism underlying the antiinflammatory and antirheumatic effect of Harpagophytum is unclear. Recent studies have indicated that the arachidonic acid cascade and prostaglandins do not appear to be involved in the antiinflammatory effect, in contrast to conventional NSAID. Current research suggests Harpagophytum has a significant antioxidant effect. As already mentioned above, free radicals are crucially involved in inflammatory reactions, and thus the antiinflammatory effect of Harpagophytum might be due to its antioxidant effect.

F. Boswellia serrata or carterii (Indian Frankincense)

Boswellia serrata or carterii (Indian frankincense) has been used as medicinal plant for more than 3000 years in ayurvedic medicine. The resin of the Indian frankincense tree has been traditionally employed in the form of an ointment for local treatment of inflammations (especially inflammations of joints), bone fractures, glandular swelling and ulcers and internally for chronic bowel disorders, oral inflammations and for hemorrhoids.

Besides numerous other substances, the frankincense gum comprises 5-8% boswellic acids. It has been shown that these acids inhibit 5-lipoxygenase and accordingly display an antiinflammatory effect. Dry extracts which can be obtained from frankincense gum comprise high percentages, e.g. 65%, of boswellic acids. Intake is recommended for all chronic inflammatory diseases with elevated leukotriene levels, inter alia for rheumatoid arthritis.

The inventive combination product comprises the above ingredients A-F either in a single dosage unit, e.g. in a capsule, or in two (e.g. omega-3 fatty acids in one, the other ingredients in another), three (e.g. omega-3 fatty acids in one, the vitamins and selenium in a second and Harpagophytum and Boswellia serrata or carterii in a third), four or five separate dosage units (e.g. capsules or tablets), which are then expediently packaged in such a way that the different dosage units which are to be taken on one day are assigned to a particular day of the week as is known for example for contraceptives. The individual active ingredients or active extracts can for an expedient dosage form be mixed with conventional pharmaceutical carriers, binders, lubricants and other excipients as is known for these active ingredients and active extracts or can easily be ascertained by the person skilled in the art of pharmaceutical technology.

### Comparative Examples with Administration of the Individual Products

All the abovementioned active ingredients or medicinal plant extracts A-F were tested singly in the treatment of rheumatoid arthritis. The following products and daily doses were used for this:

A. Omega-3 fatty acids: 8 capsules of Ameu® 500 mg. One capsule of Ameu contains 500 mg of fish body oil (fatty oil from pelagic fish) which comprises 70 mg of eicosapentaenoic acid and 50 mg of docosahexaenoic acid (total 120 mg of omega-3 fatty acids).

B. α-Tocopherol (vitamin E): 800 to 1000 I.U. once a day.

C. Ascorbic acid (vitamin C): 3 g in three 1000 mg doses.

D. Selenium: 300 μg of selenium as sodium selenite 5H₂O (Cefasel® or Selenase®) once a day.

E. Harpagophytum: 2×480 mg of dry extract once a day (extractant: ethanol 60% (V/V) (BIOCUR Arzneimittel GmbH).

F. Boswellia serrata: 3×400 mg of dry extract once a day (H 15 Gufic tablets (Gufic Chem, India)).

For these treatments and for all treatments detailed hereinafter, only patients with rheumatoid arthritis complying with the criteria of the American Rheumatism Association were included: 3 or more joints affected, morning stiffness, symmetrical arthritis of wrist and finger joints, subcutaneous arthritic swellings on joint processes or typical radiological signs (erosion, osteoporosis, etc.). A positive rheumatoid factor was not obligatory. All the treated patients had elevated C-reactive protein and frequently raised ferritin as signs of inflammation. Bacterial and parasitic causes, and allergies (including yersinia, staphylococci, streptococci, mycoplasma, Whipple's disease, borreliosis and gluten allergies) were excluded.

All the treated patients additionally showed the symptoms of pain, sensitivity to pressure or touch, limited joint mobility and restriction of physical activities on account of symptoms.

All the patients had discontinued their previous medication (morphine, ibuprofen, cortisol, etc.) at least one week before starting the treatment, or had received no medical therapy over a prolonged period because of toxic side effects of other medicaments.

The results of treatment were classified on a scale of from 1 (scarcely any alleviation of symptoms) to 10 (complete elimination of symptoms).

Results of the Treatment with the Individual Substances or Active Ingredient Extracts A-F:

Omega-3 fatty acids: After therapy for two months, a 20-35% improvement was seen with improved range of movement, diminished sensitivity to pressure or touch and reduced swelling.

Scale of 1-10: 4.5

α-Tocopherol (vitamin E): After treatment for two weeks, a slight 10% improvement was observed for joint sensitivity, but only a 5% alleviation of pain.

Scale of 1-10: 3.0

Ascorbic acid (vitamin C): After therapy for two weeks, there was a 5-10% improvement in joint sensitivity and a 2-5% reduction in joint pain.

Scale of 1-10: 2

Selenium: After 10 days to 2 weeks it was possible to observe a 3-5% reduction in joint sensitivity and a 5% improvement in pain.

Scale of 1-10: 2.5

Harpagophytum: After treatment for one week, the patients showed reduced joint pain and overall a 15-20% improvement in the symptoms.

Scale of 1-10: 3.5

Boswellia serrata: After only 2 days, a 20-25% improvement in symptoms with reduced joint sensitivity and less pain was found.

Scale of 1-10: 4.0

In summary, it can be said that the treatment with the individual substances or extracts brought about a small to moderate improvement in symptoms, although additional medication, e.g. in the form of NSAID, would scarcely be avoidable in the long term.

Examples of a treatment with the inventive combination product are indicated below. The examples describe the treatment of rheumatoid arthritis. However, the invention is not restricted to the use of the inventive combination product for this pathological condition. On the contrary it can be expected that the inventive combination product will be beneficial for all chronic inflammatory diseases in which free radicals, prostaglandins, leukotrienes and/or cytokines play a substantial role.

### EXAMPLES

As already mentioned above, only patients with rheumatoid arthritis complying with the abovementioned criteria of the American Rheumatism Association were included in the treatment with the inventive combination product.

In the examples, the following dosage ranges of products A-F were employed as required:

Omega-3 fatty acids: Minimum 8 Ameu capsules/day (equivalent to 960 mg of omega-3 fatty acids), maximum 14 Ameu capsules/day (equivalent to 1680 mg of omega-3 fatty acids)

α-Tocopherol: Minimum 800 I.U./day, maximum 2000 I.U./day

Ascorbic acid: Minimum 1 g/day, maximum 4 g/day

Selenium: Minimum 100 μg/day, maximum 300 μg/day in the form of sodium selenite 5H₂O

Harpagophytum extract: Minimum 0.96 g/day, maximum 1.92 g/day

Boswellia serrata extract: Minimum 1.2 g/day, maximum 6 g/day

30 randomly selected male and female patients from 35 to 57 years of age were given a combination of the abovementioned products A-F in the stated dosage ranges. Most of the patients had discontinued their previous medication (morphine, ibuprofen, cortisol, etc.) at least one week before starting the treatment or had received no medical therapy over a prolonged period because of toxic side effects of other medicaments (only a few patients started with the combination therapy and discontinued the previous medicaments only during the course thereof). All the patients exhibited the symptoms of pain, sensitivity to pressure or touch, morning stiffness, limitations of joint mobility and restriction of physical activities on account of symptoms.

Results of Treatment

10 patients showed a 50% improvement in the first 5 days of treatment, 15 patients experienced a 60% improvement after 10 days, and for the last 5 patients it took 4 weeks before they showed a 90% improvement. 90% of all the patients showed an 80-95% improvement which was permanent, after 4 weeks (2 patients abandoned the therapy under pressure from their families and decided to continue to take NSAID or cortisol). The symptoms of joint pain and swelling were reduced in 90% of the patients, and the limited mobility and flexibility were improved in 85% of the patients who had no ankylosis (2 patients had ankylosis). One patient with hand splints no longer required them after treatment for four weeks. In no case were disadvantageous side effects observed.

The variations at the start of the treatment are probably attributable to different dietary and health habits; the use of tobacco and alcohol and consumption of red meat might have delayed the therapeutic effect at the start of the therapy. However, scarcely any effect of different habits is noticeable after 8 weeks.

The parameter of inflammation C-reactive protein (CRP) was reduced by 95% to a normal level in 85% of the patients within four weeks. The patients who were rheumatoid factor-positive showed a reduction of 75-85% after 4 weeks.

On the scale from 1 to 10 defined above, the efficacy of the inventive combination product was 8.5-9.

The 28 patients who continued with the combination therapy all could do without additional medicaments such as cortisol, NSAID or morphine after 4 weeks.

The following are selected individual examples of the treatment with the inventive combination product.

### Example 1

A 52-year old woman with a history of rheumatoid arthritis including joint pain, especially in the hand, which made splints necessary, was treated with the above combination A-F, specifically 12 Ameu capsules (equivalent to 720 mg of omega-3 fatty acids), 800 I.U. of α-tocopherol, 3 g of ascorbic acid, 300 μg of selenium in the form of sodium selenite 5H₂O, 0.96 g of Harpagophytum extract and 2.4 g of Boswellia serrata extract. Her symptoms improved by 20% within 4 days, with a gradual advance to 85% after 4 weeks: after this she no longer required splints on her hands. The result was confirmed by her laboratory data with a reduction in the parameters of inflammation. Her CRP diminished from 14.1 to 1.6 within the normal values of <5.0. Since she had an elevated rheumatoid factor, this was likewise measured. It was within normal values after 8 weeks.

### Example 2

A 43-year old woman with a three-year history of arthritis including all the symptoms apart from radiological changes had to discontinue cortisol because of excessive water retention and weight gain. She received a combination therapy with the above products A-F. She reported an improvement in her pain after 3 days, and her morning stiffness and swelling had decreased by 50% after one week. The symptoms were reduced by 90% after 4 weeks, and she was able to resume sporting activities such as fitness exercises and cycling. The laboratory parameters correlated with the clinical results: CRP was reduced to 4 (within normal values). Her rheumatoid factor was not elevated because of the preceding cortisol therapy, and remained at normal levels after the cortisol had been eliminated from her body.

### Example 3

A 55-year old man with a 5-year history of arthritis (rheumatoid factor positive), including joint pain, swelling, limited movement, morning stiffness and ulnar deviation of the right hand, had to stop taking NSAID because of renal toxicity and was treated with the above combination of products A-F. His rheumatoid factor was 70 at the start of the treatment, and CRP was 12.5. He noticed a slight improvement after 5 days, including reduction in pain and less joint sensitivity. The laboratory data showed an improvement of CRP after 10 days, and the rheumatoid factor had fallen by 80% after 4 weeks. After 2 months, his symptoms had improved by 90% and he was able to resume normal physical activities.

### Example 4

A 38-year old multiparous woman had recently been diagnosed with rheumatoid arthritis. Bacterial and parasites were negative, the rheumatoid factor was positive and CRP was elevated. Her movement in the joints was unimpaired, but her daily activities were extremely restricted by the swelling and pain. She received treatment with the above products A-F and showed a rapid improvement after only 4 days. CRP was likewise normal after only 4 days, and the rheumatoid factor returned to normal levels after 5 weeks. Her dosage was reduced to the minimum amount of the standard daily treatment. She resumed her normal activities without symptoms within 5 days.

### Example 5

A 55-year old woman with a 10-year history of arthritis, including the symptoms of swelling, pain, limited movement and radiological changes, started with a combination treatment with the above products A-F and then discontinued cortisol and NSAID. Transient symptoms and a slight swelling occurred only on the first 3 days, and after 8 days she remained free of symptoms and no longer required cortisol or NSAID.

### Example 6

A 48-year old woman with rheumatoid arthritis who had taken cortisol, NSAID and morphine for 8 years had to discontinue cortisol because of osteoporosis and excessive water retention. She had gained 25 kg and wished to try the combination therapy because it has no side effects. Treatment with the above products A-F was initiated while her other medicaments were gradually reduced. She began to lose water, and her physical well being returned to the normal condition which existed before the other medical treatments. A limited mobility of her hands, a slight ankylosis and ulnar deviation persisted. She experienced no change in her symptoms after she had discontinued the earlier medicaments, meaning that the combination of products A-F continued to suppress her symptoms.

### Example 7

A 57-year old man with rheumatoid arthritis could not take steroids and NSAID because of renal impairment after an earlier streptococcal infection which reduced renal function. In order to control his main symptoms, inflamed joints in the hands and knees, morning stiffness and swelling, he started with a combination treatment with the above products A-F. The first improvement was in the parameter of inflammation CRP, and after 3 weeks his general stiffness and sensitivity to pressure and touch had more than 85% disappeared. After 6 weeks, the laboratory data were negative, and his symptoms were no longer noticeable.

### Example 8

A 46-year old woman with a recent diagnosis of rheumatoid arthritis received treatment with the above products A-F without any previous medical therapy. Her symptoms of joint swelling, pain, joint sensitivity and morning stiffness were reduced after only 4 days. The swelling had completely disappeared after 10 days. After 4 weeks, morning stiffness, pain and sensitivity had also been completely eliminated.

### Example 9

A 38-year old woman, mother of two children, was diagnosed with rheumatoid arthritis. Her rheumatoid factor as negative, but all the other symptoms, including joint pain, sensitivity and swelling, clearly showed an arthritic condition. They began with a treatment with the above product combination A-F, but received cortisol concurrently. The diminution in her symptoms thus could not be ascribed to the combination treatment until she ceased receiving cortisol after 2 weeks. Her symptoms remained in remission even then, which was now attributable to the combination therapy, and she was able to resume her normal daily activities and a sporting activity (jogging).

In summary, it can be said that on treatment of rheumatoid arthritis with a combination of omega-3 fatty acids, α-tocopherol, ascorbic acid, selenium, Harpagophytum procumbens and Boswellia serrata or carterii, which caused no adverse side effects in any of the people treated, entirely surprisingly an additional medical therapy with cortisol, NSAID and/or morphine is unnecessary and that, on the contrary, the symptoms and the relevant laboratory values can be alleviated or reduced to a satisfactory extent with this therapy alone. This was not predictable on the basis of the efficacy of the individual active substances or active extracts, because on treatment with these alone in most cases an additional administration of “classical” antiinflammatory drugs or analgesics cannot be avoided in the long term.

It is to be expected that the inventive combination product will also be beneficial for other chronic inflammatory diseases, such as, for example, osteoarthritis and psoriatic arthritis, in which free radicals, prostaglandins, leukotrienes and cytokines are involved.

1.-27. (canceled)

28. A natural remedy-dietary supplement combination product, wherein the product comprises omega-3 fatty acids, α-tocopherol (vitamin E), ascorbic acid (vitamin C), selenium, Harpagophytum procumbens (devil's claw) and Boswellia serrata or carterii (frankincense), with the proviso that it does not comprise a plant or plant extract of the Saxifragaceae family.

29. The combination product of claim 28, wherein the product is contained in a single dosage unit.

30. The combination product of claim 28, wherein the product is contained in two, three, four or five dosage units.

31. The combination product of claim 30, wherein the dosage units are assigned to a particular day in a package.

32. The combination product of claim 28, wherein the Harpagophytum procumbens is present as dry extract.

33. The combination product of claim 28, wherein the frankincense is present as a dry extract of Boswellia serrata.

34. The combination product of claim 28, wherein the selenium is present as one or more of sodium selenite, selenomethionine, selenoallylcysteine and selenium yeast.

35. The combination product of claim 28, wherein the omega-3 fatty acids are present as fish oil.

36. The combination product of claim 28, wherein the product further comprises one or more pharmaceutically acceptable pharmaceutical carriers.

37. The combination product of claim 28, wherein the Harpagophytum procumbens is present as dry extract, the frankincense is present as a dry extract of Boswellia serrata, the selenium is present as one or more of sodium selenite, selenomethionine, selenoallylcysteine and selenium yeast, and the omega-3 fatty acids are present as fish oil.

38. A method of treating a chronic inflammatory disorder and/or a rheumatic disorder, wherein the method comprises administering to a patient in need thereof a combination which comprises omega-3 fatty acids, α-tocopherol (vitamin E), ascorbic acid (vitamin C), selenium, Harpagophytum procumbens (devil's claw) and Boswellia serrata or carterii (frankincense) in an amount which is effective to alleviate the disorder, with the proviso that the combination does not comprise a plant or plant extract of the Saxifragaceae family.

39. The method of claim 38, wherein the disorder comprises rheumatoid arthritis (chronic polyarthritis).

40. The method of claim 38, wherein the omega-3 fatty acids are administered in a dose of from 960 to 1680 mg/day.

41. The method of claim 38, wherein the α-tocopherol is administered in an amount of from 800 I.U. to 2000 I.U. per day.

42. The method of claim 38, wherein the ascorbic acid is administered in an amount of from 1 to 4 g/day.

43. The method of claim 38, wherein the selenium is administered in an amount of from 100 to 300 μg/day.

44. The method of claim 38, wherein the selenium is administered in a form which comprises sodium selenite 5H₂O.

45. The method of claim 38, wherein the Harpagophytum procumbens is administered as a dry extract in an amount of from 0.96 to 1.92 g/day.

46. The method of claim 38, wherein the Boswellia is administered as a dry extract of Boswellia serrata in an amount of from 1.2 to 6 g/day.

47. The method of claim 39, wherein the omega-3 fatty acids are administered in a dose of from 960 to 1680 mg/day, the α-tocopherol is administered in an amount of from 800 I.U. to 2000 I.U. per day, the ascorbic acid is administered in an amount of from 1 to 4 g/day, the selenium is administered in an amount of from 100 to 300 μg/day, the Harpagophytum procumbens is administered as a dry extract in an amount of from 0.96 to 1.92 g/day, and the Boswellia is administered as a dry extract of Boswellia serrata in an amount of from 1.2 to 6 g/day.
